(19) United States
(12) Patent Application Publication
(10) Pub. No.: US 2004/0043089 A1
(43) Pub. Date: Mar. 4, 2004

(54) Buguzhi agent and composition and methods of preparing and administering the same

(76) Inventor: A-Bakr M. Rabie, Hong Kong (HK)

Correspondence Address: PENNIE AND EDMONDS, 1155 AVENUE OF THE AMERICAS, NEW YORK, NY, 100362711

(21) Appl. No.: 10/235,614

(22) Filed: Sep. 4, 2002

(51) Int. Cl. A61K 35/78

(52) U.S. Cl. 424/779; 424/777

(57) ABSTRACT

A buguzhi composition has a buguzhi agent and a carrier. The buguzhi agent can have a buguzhi extract derived from buguzhi or a buguzhi component. The carrier of the buguzhi composition can be any suitable material, such as a collagen matrix. When the buguzhi agent and the carrier are applied to a bone defect, the buguzhi agent can promote bone healing by increasing the amount of new bone formation. The buguzhi agent can be derived from the raw material of buguzhi by various processes, such as extraction. The buguzhi agent can be mixed with a suitable carrier, such as a collagen material, to form a buguzhi composition. The resulting buguzhi agent and composition can be applied directly on to a defect part of a patient.

### FIELD OF THE INVENTION

The present invention relates generally to a buguzhi agent and a buguzhi composition. In particular, the present invention relates to a buguzhi agent and a buguzhi composition that can promote bone formation. The present invention further relates to methods of preparing and administering the buguzhi agent and the buguzhi composition.

### BACKGROUND OF THE INVENTION

It is estimated that approximately 50 percent of the thousands of drugs commonly used and prescribed today are either derived from a plant source or contain chemical imitations of a plant compound (Mindell, E. R., Earl Mindell's Herb Bible, A Fireside Book (1992)). Currently, a number of medicinal formulations contain herbal components or extracts from herbs. Technically speaking an herb is a small, non-woody (i.e., fleshy stemmed), annual or perennial seed-bearing plant in which all the aerial parts die back at the end of each growing season. As the word is more generally used and as it is used herein, an herb is any plant or plant part which has a medicinal use. Thus, the term herb is also generally used to refer to the seeds, leaves, stems, flowers, roots, berries, bark, or any other plant parts that are used for obtaining extracts for healing.

Herbal medicines have been used for treating various diseases of humans and animals in many different countries for a very long period of time (see, e.g., Kessler et al., The Doctor's Complete Guide to Healing Medicines, Berkley Health/Reference Books (1996); Mindell, supra). Herbal medications are available in many forms, including capsules, tablets, or coated tablets; pellets; extracts or tinctures; powders; fresh or dried plants or plant parts; prepared teas; juices; creams and ointments; essential oils; or, as combinations of any of these forms. Herbal medicines are administered by any one of various methods, including orally, rectally, parenterally, enterally, transdermally, intravenously, via feeding tubes, and topically.

Buguzhi is a herbal medicine known as psoralea fruit, or psoralea corylifolia L. for botanical name, or fructus psoraleae for pharmaceutical name. Buguzhi has been used systemically for anti-inflammation purpose during the process of healing of traumatic injuries, such as bone fractures, or strengthening loose teeth. When being so used, however, buguzhi affects the entire body or the entire organism of a patient.

Bone graft materials have been widely used in medical and dental fields to replace bone where needed such as in the repairing of lost bones after surgery. Nevertheless, the development of bone graft materials remains a challenge in modern surgery. Currently, bone graft materials can be obtained from a patient's own bone. To use such bone graft materials, it normally requires a second surgical site on the patient's body, such as the patient's hip, to harvest the donor bone graft. The harvest procedure, however, subjects the patient to not only inconvenience but also risks associated therewith. In addition, the donor bone grafts can undergo massive resorption when being used to replace a lost bone in large defects.

Other bone graft materials, such as demineralized bone matrix (DBM), have been used to repair bones. When DBM is used alone to graft large non-regenerative defects, it can cause non-union in newly grown bones and consequently subject the same to refracture. Moreover, the use of DBM involves a high risk of disease transmission.

Based on the foregoing, there currently exists a need for osteoinductive herbal-based therapeutics which have low toxicity and demonstrably few side effects. It would be desirable to provide a buguzhi agent or a buguzhi composition that can be used locally. It would be also desirable to provide a composition that can be used as an osteoinductive agent without affecting the entire body or organism of the patient to thus minimize the inconvenience to the patient. The novel compositions of the present invention fulfill those requirements. It would be further desirable to provide a method that can promote bone formation without the need of harvesting donor bones from a patient's other body parts or implanting allografts inside a patient's body.

### SUMMARY OF THE INVENTION

The buguzhi composition of the present invention comprises a buguzhi agent and a carrier. The buguzhi agent can comprise a buguzhi extract derived from buguzhi or a buguzhi component. As used herein, the buguzhi agent itself can be in various forms. For example, the buguzhi agent can be a suspension derived from buguzhi (dried fruit of the psoralea corylifolia but this aslo embraces the seeds). The buguzhi agent can also be derived from the isolated seeds of psoralea corylifolia (bakuchi). The carrier of the buguzhi composition can be any suitable material. According to one aspect of the present invention, the carrier can be a collagen material, such as a collagen matrix. When the buguzhi agent and the carrier are applied to a bone defect, the buguzhi agent can promote bone healing by increasing the amount of new bone formation.

The present invention also relates to methods for preparing the buguzhi agent and the buguzhi composition. The buguzhi agent can be derived from buguzhi by various processes. According to one aspect of the invention, after the raw material buguzhi is gathered, it can be subjected to cleaning and/or drying. The processed buguzhi can be subjected to various methods, such as extraction, to prepare the buguzhi agent. The resulting buguzhi agent can be kept separately before administration to use on a patient. According to another aspect of the invention, the buguzhi agent can be mixed with a suitable carrier, such as a collagen material, to form a buguzhi composition.

Further, the present invention relates to methods for administering the buguzhi agent and the buguzhi composition. According to one aspect of the invention, the method for administering the buguzhi agent comprises preparing a buguzhi agent and applying the buguzhi agent and a suitable carrier to a bone defect on a patient. The application of the buguzhi agent can be prior to, simultaneously with, or subsequent to the application of the carrier. According to another aspect of the invention, the method for administering the buguzhi composition comprises preparing a buguzhi composition and applying the buguzhi composition to a bone defect on a patient.

These and other features and advantages of the present invention will be readily apparent from the following detailed description of the invention, the scope of the invention being set out in the appended claims.

### BRIEF DESCRIPTION OF THE DRAWINGS

These and other features, aspects, and advantages of the present invention will become much more apparent from the following description, appended claims, and accompanying drawings, in which:

FIG. 1 shows new bone formation at defects of the control group (carrier alone) and the experimental group (buguzhi and carrier).

FIG. 2 Panel A shows a control photograph depicting minor bone formation. Panel B shows TCM-B that contains the Chinese medicine buguzhi agent of the invention and depicts significantly more bone formation compared to the control.

### DETAILED DESCRIPTION OF THE INVENTION DEFINITIONS

The definitions below serve to provide a clear and consistent understanding of the specification and claims, including the scope intended to be given such terms.

Allogenic. The term “allogenic”, as used herein, includes tissue including bone tissue having a different gene constitution than the intended recipient, but being from the same species.

Autogenic. The term “autogenic”, as used herein, includes tissue including bone tissue from the intended recipients own body.

Biocompatible. The term “biocompatible”, as used herein, includes any material which does not provoke an adverse response in a patient. For example, a suitable biocompatible material when introduced into a patient does not itself provoke a significant immune response, and is not toxic to the patient.

Bone. The term “bone”, as used herein, includes any bone including allograft bone, autograft bone and xenograft bone, and includes bone in any form including, but not limited to, mineralized and/or demineralized bone and/or partially demineralized bone, and cancellous and/or cortical bone and/or corticocancellous, where the bone is in a form including, but not limited to, particulate bone, ground bone including but not limited to bone in the particle size range of 125 μm to about 1000 μm, preferably about 250 μm to 710 μm, bone chips, cut bone pieces including cubes, iliac crest wedges, Cloward dowels, or strips, and essentially intact bone including the patient's own bone in the patients body, and bone including for example, but not limited to, the femur, tibia, ilia, humorous, radius, ulna, ribs, whole vertebrae, mandibula, and any segment thereof.

Bone defect site. The term “bone defect site”, as used herein, refers to any site in an animal including a human, where the inducement of bone growth and/or repair, is desired.

Osteoconductive. The term “osteoconductive”, as used herein, refers to the ability of a substance to support or conduct bone growth.

Osteoinductive. The term “osteoinductive”, as used herein, refers to the ability of a substance to induce bone growth.

Patient. The term “patient”, as used herein refers to an animal including a human, who is subject to medical treatment.

Partially Demineralized Bone. The term “partially demineralized bone”, as used herein, includes any bone including cortical and/or cancellous bone, from any source including autogenic, allogenic and/or xenogenic bone, demineralized to contain less residual calcium than is present in intact natural bone (that is, demineralized to contain less than about 30 wt % residual calcium) and preferably demineralized to contain more than about 5 wt % residual calcium and less than about 30 wt % residual calcium.

Pharmaceutically Active Agent. The term “pharmaceutically active agent”, as used herein, refers to any medically useful substance including any therapeutically beneficial substance which may be used in combination with the buguzhi agent or composition of the present invention, including, but not limited to, viricides; microbicides; antibiotics; amino acids; peptides; vitamins; co-factors for protein synthesis; hormones including growth hormones; endocrine tissue; living cells including for example: stem cells, chondrocytes, bone marrow cells, and parenchymal cells; synthesizers; enzymes; angiogenic drugs including nicotine and nicotinic acid; biocompatible surface active agents; antigenic agents; cytoskeletal agents; growth factors including but not limited to: transforming growth factor, and insulin like growth factor; antitumor agents; immuno-suppressants; and permeation enhancers.

Xenogenic. The term “xenogenic”, as used herein, refers to tissue including bone tissue that is heterologous, with respect to the intended recipient, i.e. the donor and recipient are from widely separated species. For example, if the intended recipient is human, xenogenic tissue would be tissue from a species other than human.

The buguzhi agent and the buguzhi composition of the present invention can be derived from the fruit of psoralea corylifolia L. The buguzhi agent and the buguzhi composition of the present invention can also be derived from the seeds of psoralea corylifolia L (bakutchi). According to one aspect of the invention, the buguzhi composition can comprise a buguzhi agent and a suitable carrier.

The buguzhi agent and the buguzhi composition of the present invention can comprise active ingredients including, but not limited to, simple furanocoumarins such as psoralen, bergapten (5-methoxypsoralen) and xanthotoxin (8-methoxypsoralen), as well as those angular furanocoumarins, such as, for example, angelicin and isopimpinellin.

Studies on the localisation and concentration of furanocoumarins within plant tissues reveal a complex picture. Concentrations vary widely both between different species and between different tissues of the same species. High concentrations are associated in particular with the surfaces of leaves and fruits (reaching over 60% of the total in some Citrus spp.)(Zobel A M & Brown S A, J. Toxicol. Cutan. Ocul. Toxicol., 10: 223 (1991)) and sometimes with seeds, though in some species ripening is associated with an increase (eg. Angelica archangelica) and in others with a decrease (eg. Psoralea spp.) in furanocoumarin content (Zobel A M & Brown S A, Environ. Exp. Bot., 31: 447 (1991); Cappelletti M et al., J. Chem. Ecol., 18: 155 (1992)). Various forms of stress are also known to increase furanocoumarin content as are methods of drying and storage (Masuda T et al., Tennen Yuki Kagobutsu Toronkai Koen Yoshishu (Symposium on Chemistry of Natural Products), 40: 523 (1998)). Due to the wide variability of furanocoumarin concentrations reported it is difficult to give quantitative estimates for medicinal herbs. One author estimates concentrations of a number of phototoxic constituents in A. archangelica to vary from 2-20 ppm (low) while in P. corylifolia they appear to be higher (up to 1000 ppm)(Duke J A, ‘Handbook of Phytochemical Constituents of GRAS Herbs and Other Economic Plants’, CRC Press (1992)). The phototoxic constituents, common to all species, are simple (ie. minimally substituted) linear furanocoumarins—psoralen, bergapten (5-methoxypsoralen) and xanthotoxin (8-methoxypsoralen)—as well as the angular furanocoumarins—angelicin and isopimpinellin. The latter are more commonly associated with Umbelliferae and are only weakly phototoxic. Trioxsalen, peucedanin, khellin and osthenol, species of linear furanocoumarin, are also reported to exhibit weak phototoxicity (Dictionary of Natural Products (CD-ROM version 10:1), Chapman & Hall/CRC Press (2001)).

The buguzhi agent and the buguzhi composition of the present invention can also comprise active ingredients including, but not limited to, a bakuchiol, a meroterpene isolated from psoralea corylifolia or it may be corylifolinin (isobavachalcone)). Other ingredients of the buguzhi agent and the buguzhi composition of the present invention that can also comprise active ingredients include, but are not limited to, those chemical components described in Hu X. M. Zhonghua Bencao, Shanghai Scientific Technology Press, Shanghai pages 888-896 (1998), e.g., psoralen, isopsoralen (angelicin), xanthotoxin (8-methoxypsoralen), psoralidin, isopsoralidin, bakuchicin, psoralidin 2′,3′-oxide, corylidin, bavacoumestan A and B, sophoracoumestan A, asteragalin, bavachin, corylifolin, isobavachin, bavachinin, corylifolinin, isobavachalcone, bavachalcone, bavachromene, neobavachalcone, isoneobavachalcone, bakuchalcone, bavachromanol, corylin, neobavaisoflavone, corylinal, psoralenol, bakuchiol, corylifonol, isocorylifonol, p-hydroxy-benzoicacid, stigmasterol, β-sitosterol-D-glucoside, triacontane, trypsin inhibitor, palmitic acid, oleic acid, linolenic acid, and lignoceric acid.

The buguzhi agent can comprise the raw material of buguzhi, or extracts or components of the same. As used herein, components refers to: simple furanocoumarins such as psoralen, bergapten (5-methoxypsoralen) and xanthotoxin (8-methoxypsoralen), as well as those angular furanocoumarins, such as, for example, angelicin and isopimpinellin; a bakuchiol, a meroterpene isolated from psoralea corylifolia or it may be corylifolinin (isobavachalcone)); and those chemical components described above in Hu X. M. Zhonghua Bencao (1998). In one embodiment, the buguzhi agent can comprise buguzhi extracts or component having a weight percentage in the range of about 0.05 grams to about 0.5 grams of Buguzhi extract in about 0.02 grams of collagen. The amount of collagen employed in the present invention may be varied depending upon the clinical needs of the patient undergoing treatment. In another embodiment, the buguzhi extracts or component can have a weight percentage in the range of about 0.2 grams to about 0.4 grams of Buguzhi extract in about 0.02 grams of collagen.

The buguzhi agent can be in various forms. In one exemplary embodiment, the buguzhi agent can be in the form of a suspension. For example, the buguzhi agent can be a reddish brown suspension. In another exemplary embodiment, the buguzhi agent can be in the form of a solid powdery matter. The buguzhi agent can be adapted so that it can be mixed with a suitable carrier to form a buguzhi composition as discussed below. It will be appreciated that other forms of the buguzhi agent are also within the scope of the invention. In particular, the buguzhi agent can be mixed with other chemicals such as statins or bone morphogenic proteins with different matrix carriers.

For example, the buguzhi agent of the present invention can be administered with other peptides and/or proteins including, but not limited to, bone morphogenic proteins such as BMP-2, BMP-3, BMP-4, BMP-5, BMP-6 (Vgr-1), BMP-7 (OP-1), BMP-8. BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. The buguzhi agent of the present invention can be administered with other peptides and/or proteins including, but not limited to, growth factors (FGF, FGF-1, FGF-2, VEGF, Endotherial Mitogenic Growth Factors, and epidermal growth factors, transforming growth factor α and β, platelet derived endothelial growth factor, platelet derived growth factor, tumor necrosis factor α, hepatocyte growth factor and insulin like growth factor), transcription factors, proteinkinases, CD inhibitors, thymidine kinases.

The buguzhi agent of the present invention can be administered with other biologically active material that include, for example, but not limited to, non-genetic therapeutic agents, such as: anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid, amlodipine and doxazosin; anti-inflammatory agents such as glucocorticoids, betamethasone, dexamethasone, prednisolone, corticosterone, budesonide, estrogen, sulfasalazine, and mesalamine; anti-neoplastic/antiprolifcrative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, taxol and its analogs or derivatives; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides; growth factor receptors, transcriptional activators, and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; and agents which interfere with endogenous vasoactive mechanisms; anti-oxidants, such as probucol; antibiotic agents, such as penicillin, cefoxitin, oxacillin, tobranycin; angiogenic substances, such as acidic and basic fibrobrast growth factors, estrogen including estradiol (E2), estriol (E3) and 17-Beta Estradiol; and drugs for heart failure, such as digoxin, beta-blockers, angiotensin-converting enzyme (ACE) inhibitors including captopril and enalopril.

The carrier in the buguzhi composition can be various materials, which are capable of reconstituting the buguzhi agent. Factors, such as the intended application of the buguzhi composition, can be used as a consideration in determining what suitable carrier to use in the buguzhi composition. For example, a suitable carrier can be selected to be capable of disseminating the active buguzhi extracts or components regularly and slowly after the buguzhi agent is administered to a patient. Exemplary suitable carriers can include, but not limited to, collagen, hyaluronate, polylactic acid, porous bone mineral (for example, BIO-OSS collagen (cancellous natural bone mineral granules with collagen fibers). In one embodiment, the active buguzhi extracts or components with a suitable carrier are released in a regular and slow rate to thus promote bone healing after the buguzhi extracts or components are administered to a patient, wherein the release is at a rate sufficient enough to promote bone formation in said patient.

In an exemplary embodiment, the carrier can comprise a collagen material, such as a collagen matrix. When mixed with the buguzhi agent, the collagen matrix carrier is capable of disseminating the active buguzhi extracts or components regularly and slowly to thus promote bone healing. Exemplary collagen matrix carriers can include, but are not limited to, bovine collagen, human collagen and collagen derived from rabbits. In one embodiment, the active buguzhi extracts or components with a suitable collagen carrier matrix are released in a regular and slow rate to thus promote bone healing after the buguzhi agent is administered to a patient, wherein the release is at a rate sufficient enough to promote bone formation in said patient.

The buguzhi composition so formed is capable of promoting new bone formation, such as by increasing the amount of newly formed bone as will be discussed in greater detail below.

Various collagens are contemplated for use in the buguzhi composition of the present invention. For example, collagens are a family of proteins with a well determined triple helical configuration. The family of collagens comprise Type I to type XIX collagens. Among these proteins, collagen Type I is most prevalent, constituting approximately 25% of the body's proteins and 80% of the connective tissues' proteins. The primary source of type I collagen is tendon, skin, bone, and ligament.

Collagen Type I polymerizes to form aggregates of fibers and bundles. Collagen are continuously remodeled in the body by degradation and synthesis. Collagen Type I is degraded only by a specific enzyme—collagenase, and is resistant to any non-specific proteolytic degradation. Collagen is a weak antigen and most of its antigenicity resides in the non-helical terminals of the molecule. These terminals may be removed by enzymes such as pepsin. Indeed, collagen's weak antigenicity and its relative resistance to degradation make collagen a good candidate as a building material of various implantable devices.

Both human and animal tissues may be used to isolate the collagen. In general, animal tissues are preferred due to easy availability in fresh forms from local slaughter houses. A molecular solution of type I collagen can be prepared from a connective tissue rich in this protein and the molecular collagen can then be reassembled into fibrils which can then combine to form a collagen matrix. Collagen matrices can be molded in vitro into numerous implantable devices such as, for example collagen sheets, collagen tubes, etc.

Freeze-dried collagen matrix may be prepared using techniques well known to those of skill in the art. For example, a review of the preparation of collagen can be found in “Methods in Enzymology,” vol. 82, pp. 33-64, 1982, the entire contents of which are incorporated by reference in their entirety. The freeze-dried collagen matrix is then subjected to a crosslinking process to introduce additional intermolecular crosslinks to stabilize the form of the collagen matrix. The crosslinking is carried out by means well known in the art. Any reagents which can chemically react with amino groups, hydroxyl groups, guanidino groups, carboxyl groups that can link the side chains of different collagen molecules may be used to crosslink the collagen matrix. This can be accomplished with chromium sulfate, formaldehyde, glutaraldehyde, carbodiimide, adipyl chloride, hexamethylene diisocyanate and the like. The rate of in vivo resorption of the collagen is dependent upon the degree of intermolecular crosslinking in the collagen matrix. Factors controlling the extent of crosslinking are the type and concentration of the crosslinking agent; the pH, time and the temperature of incubation in the liquid phase; or the vapor pressure of the crosslinking agent, time, temperature and the relative humidity when carrying out crosslinking in the vapor phase.

In one embodiment of the present invention, if the collagen matrix is additionally intended to function as a medicinal delivery vehicle, then in addition to the type I collagen, medicinal additives may optionally be included in the dispersion of the buguzhi collagen matrix composition, such as antibiotics, thrombin, polysaccharides such as hyaluronic acid, chondroitin sulfates, alginic acids, chitosan and the like, growth factors such as epidermal growth factors, transforming growth factor beta (TGF-.beta.) and the like, glycoproteins such as fibronectin, laminin, and the like, type II through type XIX collagens, and mixtures thereof.

Thus, in one embodiment, the collagen in the collagen matrix material of the buguzhi composition is preferably type I, but may be any other type of collagen. In another embodiment, the matrix material may optionally include two or more types of collagen (e.g., selected from types I, II, III, IV, V, VI, VII, VIII, IX, X, XI, XII, XIII, XIV, XV, XVI, XVII, XVIII, and XIX), as well as any additional components that impart desirable characteristics to the resulting matrix: e.g., agarose, alginate, fibronectin, laminin, hyaluronic acid, heparan sulfate, dermatan sulfate, chondroitin sulfate, sulfated proteoglycans, fibrin, elastin, or tenascin.

According to one aspect of the invention, the buguzhi agent can be mixed with a suitable carrier to form a buguzhi composition. The proportion of the buguzhi agent to the carrier can vary depending on the applications of the buguzhi composition.

In one exemplary embodiment, the buguzhi agent can have a weight percentage in the range of about 0.05 grams to about 0.5 grams of buguzhi extract in about 0.02 grams of collagen. In another exemplary embodiment, the buguzhi agent can have a weight percentage in the range of about 0.2 grams to about 0.4 grams of Buguzhi extract in about 0.02 grams of collagen. It will be appreciated that other weight percentages of the buguzhi agent are still within the scope of the present invention.

In one embodiment, the buguzhi composition can comprise the buguzhi agent and a collagen carrier as described above. The buguzhi agent and the collagen carrier can have various proportions. In one exemplary embodiment, the ratio of the buguzhi agent to the collagen carrier can be in the range of about 2.5:1 to about 25:1. In another exemplary embodiment, the ratio of the buguzhi agent to the collagen carrier can be in the range of about 10:1 to about 20:1. In general, the stated ranges for the ratio of the buguzhi agent to the collagen carrier are applicable regardless of the source or type of collagen carrier employed in the buguzhi composition of the present invention.

According to another aspect of the invention, the buguzhi agent and the buguzhi composition can be prepared by various methods, such as by extraction, boiling with water, dissolved in organic solvents such as alcohols, separation techniques using chromatological methods, and super critical CO₂ fluid extraction, etc. In one embodiment, the buguzhi agent can be prepared by extracting a raw material of buguzhi. For example, the raw material can be first collected. Optionally, such raw material can be subjected to cleaning or drying or both before the extraction. The raw material can then be processed to obtain the buguzhi agent. For example, and not by way of limitation, buguzhi powder was boiled with water of injection and filtered. The filtrate was added with water of injection to the desired concentration and the buguzhi preparation was then sterilized.

In an alternative embodiment, the buguzhi agent can be prepared by immersion in alcohols such as ethanol, wherein the immersion time in ethanol is sufficiently long enough to release the buguzhi agent from its source. It will be appreciated that other methods of preparing the buguzhi agent are still within the scope of the present invention.

Further, the extracts of plants belonging to Psoralea corylifolia may be obtained advantageously from the adult form of these plants, but can also be extracted from the dried mature fruit. The extracts of plants belonging to Psoralea corylifolia may be obtained from the seeds and/or roots of these plants.

When extracting the extracts of plants belonging to Psoralea corylifolia, any methods generally used when extracting plant-derived extracts can be used. That is, a plant belonging to Psoralea corylifolia may be used for solvent extraction in the fresh state or, if desired, dried, then used as it is or pulverized. The solvent used for extraction may be any conventional solvent generally usable when extracting the ingredients of a plant from the plant and is not particularly limited.

For example, the extract derived from the Psoralea corylifolia plant material, such as buguzhi fruit), Bakuchi (seeds), leaves and/or the roots of Psoralea corylifolia, may be a water/water-miscible organic solvent extract. In any event, the organic solvent ultimately employed must not disrupt the active ingredient of the buguzhi agent of the present invention. For example, some components such as psoralen are only sparingly soluble in water. The ratio of water to water-miscible organic solvent is generally in the order of 0.5% to 70% v/v water-miscible organic solvent. The water-miscible organic solvent is preferably a C₁₋₄ water-miscible organic solvent (such as methanol, ethanol, propanol, propylene glycol, erythrite, butanol, butanediol, acetonitrile, ethyleneglycol, glycidol, glycerol dihydroxyacetone or acetone). The extract in this regard is prepared by exposing the Psoralea corylifolia plant material to the water/water-miscible solvent mix. The exposure time in general terms is indirectly proportional to the temperature of the mixture. The temperature of the mix may range, for example, from an ambient temperature to boiling temperature. Exposure time may be between, for example, one hour to, for example, several weeks. Undesired components may be removed from the extract to give a final Psoralea corylifolia plant extract as utilised herein by standard procedures. Examples include chromatographic techniques, such as preparative high performance liquid chromatography (HPLC) using UV detection. Examples of chromatographic media include inorganic materials (such as porous silica, controlled poreglass hydroxy apatite, fluorapatite, aluminium oxide), composite materials (such as coated silica, coated polystyrene) and synthetic polymers (polyacrylamide, polymethacrylate, and polystyrene). The solvent phase for chromotographic separation may be an organic solvent such as methanol, ethanol, propanol, butanol, penatol, acetone, butanone, chloroform, dichloromethane, dichloroethane, dichlorobutane, ethylacetate, ether or dimethyl sulphoxide, which may be used to dissolve the extract.

In another embodiment, the buguzhi composition can be prepared by mixing a buguzhi agent with one or more pharmaceutically acceptable carriers, excipients, auxiliaries, and/or diluents. The mixture can be prepared by mixing together the correct ratio of buguzhi extract and collagen together, and optionally formulating the extract with one or more pharmaceutically acceptable carriers, excipients, auxiliaries, and/or diluents. For example, the mixture can be prepared by simple mixing of buguzhi extract with collagen matrix. In an alternative embodiment, the buguzhi composition can be prepared by mixing buguzhi extract with porous bovine bone mineral or hyaluronate carrier, and then optionally formulating the extract with one or more pharmaceutically acceptable carriers, excipients, auxiliaries, and/or diluents. It will be appreciated that other methods of preparing the buguzhi composition are still within the scope of the present invention.

According to another aspect of the invention, the buguzhi agent and the buguzhi composition can be administered locally to a body part of a patient in need thereof. In one embodiment, a predetermined amount of the buguzhi agent or the buguzhi composition can be applied to a defect body part of the patient in need thereof. In an exemplary embodiment, the application of the buguzhi agent can be prior to, concurrently with, or subsequent to the application of a suitable carrier.

The amount of the buguzhi agent or the buguzhi composition administered can vary depending on various factors, such as the type and degree of the defect to which the buguzhi agent or the buguzhi composition is to be applied. In general, what constitutes an effective amount of the compositions of the present invention will depend upon a number of factors, such as specific mode of administration, the bone condition being treated, the condition of the patient and the judgement of the health care giver. Examples of suitable dosages of extracts are about 0.1 mg to about 200 mg per day, such as in the order of 1.5 mg/kg (body weight)/day. In an exemplary embodiment, the amount of the buguzhi agent applied to the defect body part can be in the range of about 0 grams/mm² to about 5 grams/mm². In another exemplary embodiment, the amount of the buguzhi agent applied to the defect body part can be in the range of about 0 grams/mm² to about 0.2 grams/mm². In another exemplary embodiment, the amount of the buguzhi agent applied to the defect body part will be about 0.22 grams/mm². It will be appreciated that other amounts of the buguzhi agent amount is also within the scope of the invention. In any event, the total amount of the buguzhi agent applied to a patient in need thereof is on the order of approximately 10 grams.

The buguzhi agent and the buguzhi composition of the invention can have various applications. For example, the buguzhi agent and the buguzhi composition can be used in medical and dental fields, such as to treat trauma, to repair bones, and to treat cleft palate. According to one aspect of the invention, the buguzhi agent and the buguzhi composition can be applied locally, such as to a body part of a patient.

In one embodiment, the buguzhi agent and the buguzhi component can be used as an osteoinductive material. In an exemplary embodiment, the buguzhi agent can be applied locally, such as to a bone defect of a patient, with a suitable carrier, such as a collagen material. When the buguzhi agent is applied to a bone defect with such a collagen carrier, the buguzhi agent is capable of promoting bone healing by increasing the amount of new bone formation. The buguzhi agent and buguzhi composition of the invention can treat any bone defects regardless of the age of the patient.

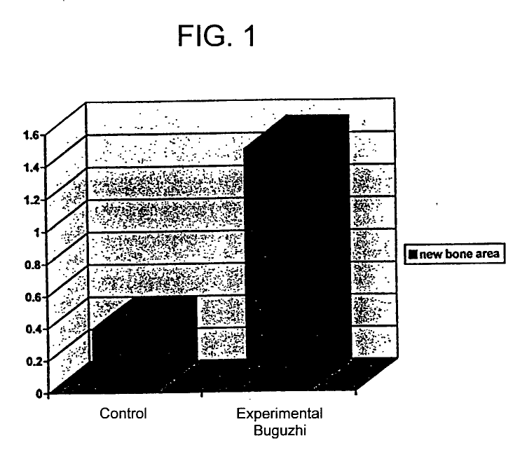

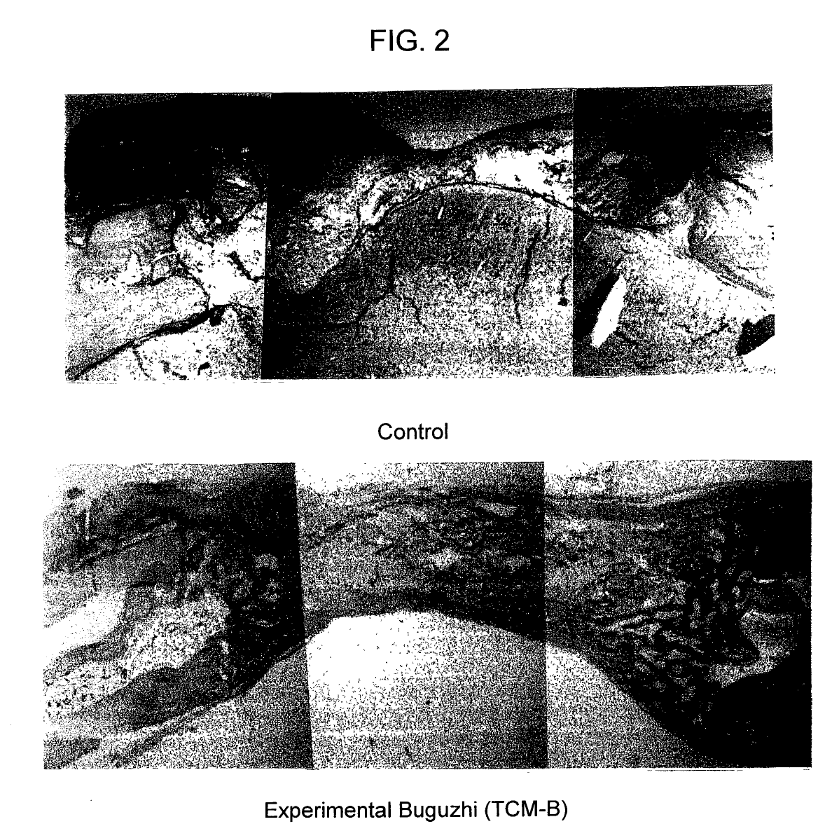

In another exemplary embodiment, the buguzhi agent and the buguzhi composition can be used as a bone graft material. The buguzhi agent, when mixed with an appropriate carrier, such as collagen material, can induce new bone formation and accelerate bone healing. Accordingly, the use of the buguzhi agent or the buguzhi composition can improve surgical conditions and results, such as eliminating the use of allografts and the need to harvest donor bones. In an exemplary embodiment, the buguzhi composition of the invention can increase the amount of new bone formation by about 400% comparing to that when the carrier is used alone, such as shown in FIG. 1. In FIG. 2, the picture at the top represents the control group (carrier alone); and the picture in the middle represent the group treated by the traditional Chinese medicine buguzhi and carrier (TCM-B). The new bone is forming next to the bone ends of the defect It is evident that there is more bone forming in the TCM-B group compared to the control group.

In a further embodiment, the buguzhi agent and the buguzhi composition can not only increase the amount of new bone formation thus improving and accelerating the bone healing but also eliminate or decrease the amount of bone graft required for the treatment of large bone defects. In this embodiment, more bone formation would mean that in this instance less bone graft material amounts would be required.

In yet a further embodiment, the buguzhi agent and the buguzhi composition of the present invention can also be used in the treatment of cleft palate, bone defect, bone fracture, bone loss in periodontal disease, as an adjunct to bone grafts and in implant surgery.

As set forth above compositions according to the present invention may include one or more pharmaceutically acceptable carriers. The carriers are selected so as to be acceptable in the sense of being ingredients in the composition and must not be deleterious to the patient. The carriers may be solid or a liquid, or both, and may be formulated with the extract as a unit-dose, for example a tablet, which may contain from 0.5% to 59% by weight of the active compound or up to 100% by weight to the active compound. Compositions may be prepared by any of the well known techniques of pharmacy, (for example admixing the components, optionally including excipients, diluents (for example water) and auxiliaries as are well known in the pharmaceutical field.

The compositions according to the invention may include one or more active agents, such as vitamins (for example, Vitamin A, Vitamin B group, Vitamin C, Vitamin D, Vitamin E and Vitamin K), minerals (for example, magnesium, iron, zinc, calcium and manganese in the form of pharmaceutically acceptable salts), chemotherapy agents including anti-multi-drug resistant compounds (for example, alkylating agents, anti-metabolites, vinca alkaloids, antibiotic cytotoxics, hormonal antineoplastic agents, and synthetic cytotoxics), immune stimulators (for example, any interferon, interleukin, and growth hormones/growth factors), anti-oxidants, statins, bone morphogenic proteins, and various growth factors referred to above.

The compositions of the invention include those suitable for oral, rectal, optical, buccal (for example sublingual), parental (for example subcutaeous, intramuscular, intradermal and intravenous) and transdermal administration. The most suitable route in any given case will depend on the nature and severity of the condition being treated and the state of the patient in need of such treatment.

Compositions of the invention suitable for oral administration may be presented in discrete units, such as capsules, cachets, lozenges, or tablets, each containing a predetermined amount of the extract; as a powder or granules; as a solution or a suspension in an aqueous or non-aqueous liquid; or as an oil-in-water or water-in-oil emulsion. Such compositions may be prepared by any suitable method of pharmacy which includes the step of bringing into association the active compound and one or more suitable carriers (which may contain one or more accessory ingredients as noted above). In general the compositions of the invention are prepared by uniformly and intimately admixing the extract with a liquid or finely divided solid carrier, or both, and then, if necessary, shaping the resulting mixture. For example, a tablet may be prepared by comprising or moulding a powder or granules containing the extract, optionally with one or more accessory ingredients. Compressed tables may be prepared by compressing in a suitable machine, the extracts in the form of a powder or granules optionally mixed with a binder, lubricant, inert diluents, and/or surface active/dispersing agent(s). Moulded tablets may be made by moulding, in a suitable machine, the powdered compound moistened with an inert liquid binder.

The buguzhi agent of the present invention can also be formulated for controlled release by coating the buguzhi agent with a sustained-release coating. The term “sustained-release coating” refers to a coating made of one or more materials that allows for the slow release of the buguzhi agent over time. Preferably, the sustained-release coating is a pH-independent layer, i. e., a coating that has a defined permeability that is not influenced by pH. The term “pH-independent layer” means that the difference, at any given time, between the amount of buguzhi agent released at, e.g., pH 1.6, and the amount released at any other pH, e.g., pH 7.2, when measured using a specific method, such as, for example, the USP Paddle Method at 100 rpm in 900 ml aqueous buffer, is 10% (by weight) or less.

Any sustained-release coating known to those of ordinary skill in the art can be used in the oral dosage form of the invention. Sustained-release coatings are well known in the art (See, e.g., Remingtons Pharmaceutical Sciences, 18^{th} ed. Mack Publishing Co., Easton, Pa., 1990, p. 1670). Typically, the sustained-release coating comprises a water- insoluble material, such as a wax or a wax-like substance, fatty alcohol, shellac, zein, hydrogenated vegetable oil, water insoluble cellulose, polymer of acrylic and/or methacrylic acid, or any other slowly digestible or dissolvable solid known in the art. The coating formulations useful in the present invention should be capable of producing a strong, continuous film that is smooth and elegant, capable of supporting pigments and other coating additives, non-toxic, inert, and tack-free. Generally, the film coat is applied to first composition, for example when in the form of a tablet or a granule, to achieve a weight gain level from about 2 to about 25 percent. However, the film coat may be lesser or greater depending upon the physical properties of the buguzhi agent included in the formulation and the desired release rate.

Alternatively, the buguzhi agent can be dispersed in a controlled-release matrix. The phrase “controlled-release matrix,” as used herein means a matrix that slowly releases the buguzhi agent over time. Any controlled-release matrix can be used in the oral dosage form of the invention. Certain controlled-release matrices are known for oral formulations (See, e.g., Remingtons Pharmaceutical Sciences, 18^{th} ed. Mack Publishing Co., Easton, Pa., 1990, p. 1684-1685). Other examples of useful controlled-release matrices are described in U.S. Pat. No. 6,143,328 to Heafield et al.; U.S. Pat. No. 6,063,405 to Drizen et al.; U.S. Pat. No. 5,462,747 to Radebaugh et al.; U.S. Pat. No. 5,451,409 to Rencher et al.; U.S. Pat. No. 5,334,392 to Cuine et al.; and U.S. Pat. Nos. 5,266,331, 5,549,912, 5,508,042, 5,656,295, 5,324,351, 5,356,467, and 5,472,712, each to Oshlack et al., the contents of which are expressly incorporated herein by reference thereto.

Suitable carriers for the present invention, other than the collagen-based carriers already exemplified above, may be fillers, such as sugars, for example lactose, saccharose, mannitol or sorbitol, cellulose preparations and/or calcium phosphates, for example tricalcium phosphate or calcium hydrogen phosphate, and also binders, such as starch pastes using, for example, corn, wheat, rice or potato starch, gelatin, tragacanth, methylceullose and/or polyvinylpyrrolidone, and, if desired, disintegrators, such as the above-mentioned starches, also carboxymethyl starch, cross linked polyvinyl pyrrolidone, agar or alginic acid or a salt thereof, such as sodium alginate. Excipients may be flow conditioners and lubricants, for example silicic acid, talc, stearic acid or salts thereof, such as magnesium or calcium stearate, and/or polyethylene glycol. Dragee cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents or solvent mixtures, or, for the preparation of enteric coatings, solutions of suitable cellulose preparations, such as acetylcellulose phthalate or hydroxypropylmethylcellulose phthalate. Dyes or pigments may be added to the tablets or dragee coatings, for example for identification purposes or to indicate different doses of active ingredients.

Other orally administrable pharmaceutical compositions are dry-filled capsules made, for example, of gelatin, and soft, sealed capsules made of gelatin and a plasticiser, such as glycerol or sorbitol. The dry-filled capsules may comprise the extracts in the form of granules, for example in admixture with fillers, such as lactose, binders, such as starches, and/or glicants, such as talc or magnesium stearate, and, where appropriate, stabilisers. In soft capsules, the extract is preferably dissolved or suspended in suitable liquids, such as fatty oils, paraffin oil or liquid polyethylene glycols, to which stabilisers may also be added.

Compositions of the present invention suitable for parenteral administration conveniently comprise sterile aqueous preparations of the extracts, which preparations are preferably isotonic with the blood of the intended patient in need thereof. These preparations are preferably administered intravenously, although administration may also be effected by means of subcutaneous, intramuscular, or intradermal injection. Suitable compositions include water soluble extracts and also suspensions of the active ingredient, such as corresponding oily injection suspensions, there being used suitable lipophilic solvents or vehicles, such as fatty oils, for example sesame oil, or synthetic fatty acid esters, for example ethyl oleate or triglycerides, or aqueous injection suspensions comprising viscosity-increasing substances, for example sodium carboxymethylcellulose, sorbitol and/or dextran, and, where appropriate, also stabilisers. As an example. compositions may conveniently be prepared by admixing the extracts with water or a glycine buffer and rendering the resulting solution sterile and isotonic with the blood. Injectable formulations according to the invention may contain from 0. 1% to 60% w/v of the extract and may, for example, be administered at a rate of 0.1 ml/minute/kg. Further details on techniques for formulation and administration may be found in the latest edition of Remington's Pharmaceutical Sciences (Maack Publishing Co., Easton, Pa.).

Compositions suitable for topical administration to the skin preferably take the form of an ointment, cream, lotion, paste, gel, spray, aerosol, or oil. Carriers which may be used include petroleum jelly, lanoline, polyethylene glycols, alcohols, and a combination of two or more thereof. The extract is generally present at a concentration of from 0.1% to 30% weight/weight, for example from 0.5% to 10% weight/weight.

### EXAMPLES

Tables 1 and 2 below illustrate different results of new bone formation using collagen matrix alone (the Control Group) and using both buguzhi and collagen carrier (the TCM-B Group), respectively. Table 1 shows the amount (mm²) of new bone formed at five defects grafted with a collagen matrix obtained from tendon collagen. Table 2 shows the amount (mm²) of new bone formed at five defects grafted with the buguzhi agent and a collagen carrier obtained from tendon collagen. Tables 1 and 2 also show mean, standard deviation (SD), and ANOVA (degree of freedom [df], F, and P) of five regions (A to E) within each defect. In this experiment, the first group shown in Table 1 measuring new bone formation at defects grafter with a collagen carrier, acts as the control for the buguzhi agent plus collagen carrier (Table 2).

TABLE 1
New Bone Formation (mm²) at Defects Grafted with a Collagen
Carrier (the Control Group)

| Region | Defect 1 | Defect 2hu a | Defect 3 | Defect 4 | Defect 5 |
|---|---|---|---|---|---|
| A | 0.68 | 0.64 | 0.18 | 0.12 | 0.41 |
| | 0.74 | 0.82 | 0.18 | 0.11 | 0.42 |
| B | 0.57 | 0.68 | 0.15 | 0.31 | 0.29 |
| | 0.51 | 0.71 | 0.16 | 0.38 | 0.34 |
| C | 0.70 | 0.85 | 0.35 | 0.23 | 0.31 |
| | 0.70 | 0.96 | 0.32 | 0.18 | 0.35 |
| D | 0.58 | 0.86 | 0.20 | 0.22 | 0.07 |
| | 0.74 | 0.95 | 0.21 | 0.24 | 0.08 |
| E | 0.60 | 0.51 | 0.06 | 0.15 | 0.11 |
| | 0.80 | 0.62 | 0.06 | 0.13 | 0.09 |
| Mean | 0.662 | 0.76 | 0.187 | 0.145 | 0.247 |
| SD | 0.09223 | 0.1503 | 0.09393 | 0.07412 | 0.1431 |
| Df | 4, 5 | 4, 5 | 4, 5 | 4, 5 | 4, 5 |
| F | 1.379 | 6.494 | 179.23 | 34.071 | 96.734 |
| P | 0.3603 | 0.0324 | <0.0001 | 0.0008 | <0.0001 |

^{a}This relatively high level of new bone formation may be due to the graft having possibly being disturbed in the experimental period. It is possible to have variations in the healing response.

TABLE 2
New Bone Formation (mm²) at Defects Grafted with the Buguzhi Agent
and a Collagen Carrier (the TCM-B Group)

| Region | Defect 1 | Defect 2 | Defect 3 | Defect 4 | Defect 5 |
|---|---|---|---|---|---|
| A | 0.88 | 0.46 | 1.27 | 2.39 | 3.51 |
| | 0.81 | 0.56 | 1.40 | 2.41 | 4.11 |
| B | 1.13 | 0.59 | 1.66 | 1.83 | 1.23 |
| | 1.01 | 0.58 | 1.66 | 1.94 | 1.65 |
| C | 1.40 | 0.31 | 1.57 | 2.73 | 1.75 |
| | 1.35 | 0.28 | 1.69 | 2.89 | 2.13 |
| D | 1.27 | 1.08 | 1.25 | 2.06 | 1.90 |
| | 1.06 | 1.09 | 1.33 | 2.25 | 2.02 |
| E | 1.55 | 0.86 | 0.75 | 2.16 | 1.28 |
| | 1.14 | 0.75 | 0.65 | 2.21 | 1.17 |
| Mean | 1.16 | 0.656 | 1.323 | 2.287 | 2.075 |
| SD | 0.2340 | 0.2862 | 0.3679 | 0.33 16 | 0.9825 |
| Df | 4, 5 | 4, 5 | 4, 5 | 4, 5 | 4, 5 |
| F | 4.013 | 78.216 | 62.597 | 31.012 | 9.460 |
| P | 0.0799 | 0.0001 | 0.0002 | 0.0010 | 0.0010 |

In a comparison between the combination of buguzhi and collagen (TCM-B) versus collagen carrier alone (Control), Tables 1 and 2 indicate that the combination of buguzhi with collagen carrier resulted in the formation of significantly more bone formation than that of collagen alone.

A comparative analysis of the data obtained for the TCM-B and the Control groups is presented in Table 3.

TABLE 3
Summary of Data for Comparison of TCM-B and Control Groups

| Parameter | TCM-B Group | Control Group |
|---|---|---|
| Mean (mm²) | 1.5002 | 0.4002 |
| Number of sections | 50 | 50 |
| Standard deviations (mm²) | 0.7853 | 0.2826 |
| Standard error (mm²) | 0.1111 | 0.03997 |
| Minimum (mm²) | 0.28 | 0.03 |
| Maximum (mn²) | 4.11 | 0.96 |
| Median (mm²) | 1.34 | 0.33 |
| Lower 95% confidence interval | 1.277 | 0.3198 |
| Upper 95% confidence interval | 1.724 | 0.4866 |

The mean difference between TCM-B and Control Groups was 1.1 mm². Using an unpaired t test the 95% confidence interval of the difference was 0.8640 to 1.336 mm². In this test, the Welch's approximate t=9.319 with 61 degrees of freedom. The two-tailed P value is<0.0001 which is considered extremely significant. The test does not assume equal variances.

### EQUIVALENTS

It will be appreciated that the various features described herein may be used singly or in any combination thereof. Therefore, the present invention is not limited to only the embodiments specifically described herein. While the foregoing description and drawings represent a preferred embodiment of the present invention, it will be understood that various additions, modifications, and substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiment is therefore to be considered in all respects as illustrative and not restrictive, the scope of the invention being indicated by the appended claims, and not limited to the foregoing description.

What is claimed is:

1. A buguzhi agent comprising an extract or a component of buguzhi having a weight percentage from about 0.5% to about 2%.

2. The buguzhi agent of claim 1, wherein the weight percentage is from about 0.2% to about 5%.

3. A buguzhi composition comprising a buguzhi agent and a carrier, wherein the buguzhi agent has a weight percentage from about 85% to about 95%.

4. The buguzhi composition of claim 3, wherein the buguzhi agent comprises an extract or a component of buguzhi.

5. The buguzhi composition of claim 3, wherein the buguzhi agent has a weight percentage from about 70% to about 97%.

6. The buguzhi composition of claim 3, wherein the carrier has a weight percentage from about 3% to about 28%.

7. The buguzhi composition of claim 3, wherein the carrier has a weight percentage from about 3% to about 28%.

8. The buguzhi composition of claim 3, wherein the carrier comprises a collagen carrier.

9. The buguzhi composition of claim 3, wherein the carrier comprises a collagen matrix.

10. A method of preparing a buguzhi agent, comprising: collecting a raw material of buguzhi; and
processing the raw material to obtain a buguzhi agent.

11. The method of claim 10, wherein the buguzhi agent obtained has a weight percentage from about 0.5% to about 2%.

12. The method of claim 10 further comprising cleaning the raw material of buguzhi after collecting the same.

13. The method of claim 10 further comprising drying the raw material of buguzhi after collecting the same.

14. The method of claim 10, wherein the raw material of buguzhi is processed by extraction.

15. The method of claim 10, wherein the raw material of buguzhi is processed to form a suspension of the buguzhi agent.

16. A method of preparing a buguzhi composition, comprising: collecting a raw material of buguzhi; processing the raw material to obtain a buguzhi agent; and mixing the buguzhi agent with a carrier.

17. The method of claim 16, wherein the ratio of the buguzhi agent to the carrier is in the range of about 70% to about 97%.

18. A method of administering a buguzhi agent, comprising: applying a predetermined amount of the buguzhi agent to a defect body part of a patient; and applying a predetermined amount of a carrier to the same defect body part of the patient.

19. The method of claim 18, wherein applying the buguzhi agent and the carrier in a weight percentage ratio of about 70% to about 97%.

20. The method of claim 18, wherein applying the buguzhi agent in an amount of about 0.2 grams/mm².

21. The method of claim 18, wherein applying the buguzhi agent in an amount of about 0.2 grams/mm².

22. A method of administering a buguzhi composition, comprising applying a predetermined amount of the buguzhi composition to a defect body part of a patient.

23. The method of claim 22, wherein applying the buguzhi composition in an amount of about 0.22 grams/mm².

24. The method of claim 22, wherein applying the buguzhi composition in an amount of about 0.22 grams/mm².
